(19) United States
(12) Patent Application Publication
(10) Pub. No.: US 2021/0244445 A1
(43) Pub. Date: Aug. 12, 2021

(54) OSTEOSYNTHESIS ASSEMBLY KIT AND USE OF AN ECCENTRIC CONNECTOR

(71) Applicant: UNIVERSITÄT BASEL, Basel (CH)

(72) Inventor: Stefan STÜBINGER, Binningen (CH)

(21) Appl. No.: 16/972,806

(22) PCT Filed: Jun. 4, 2019
(86) PCT No.: PCT/EP2019/064451

§ 371 Date: Dec. 7, 2020

(30) Foreign Application Priority Data
Jun. 8, 2018 (EP) 18176855.7

(51) Int. Cl. A61B 17/68; A61B 17/17

(52) U.S. Cl. CPC A61B 17/688; A61B 17/17

(57) ABSTRACT

An osteosynthesis assembly kit includes a bone implant, in particular an artificial bone implant, and at least two two-part eccentric connectors.

### BACKGROUND AND SUMMARY OF THE INVENTION

Exemplary embodiments of the present invention relate to an osteosynthesis assembly kit and the use of an eccentric connector.

The use of osteosyntheses, e.g., for the attachment of an artificial bone plate to a bone, currently takes place in various ways. On the one hand, the bone plate is placed over the bone at the edge or connecting bridges are placed between the bone implants which are tightened with screws. In both cases, a protrusion through the bridges or the bone plates is visible.

In another case, a bone plate can be connected to the skull cap with laterally inserted screws, so-called bone screws. In this case, very precise craftsmanship is required. In addition, situations can occur where there is not enough bone material to anchor the screw.

Taking the above-mentioned preliminary considerations into account, corresponding osteosynthesis assembly kits are therefore very expensive.

Accordingly, exemplary embodiments of the present invention provide a stable and easy-to-assemble osteosynthesis assembly kit.

The present invention relates to an osteosynthesis assembly kit. It comprises at least one bone implant. This may be an artificial bone implant, e.g., made of magnesium oxide, PEEK (polyetheretherketone), metal (e.g., stainless steel or titanium) or resorbable components (e.g., polylactide). This artificial bone implant can preferably be made according to the specific needs of the patient. However, a bone implant in a further embodiment variant can also be an autologous bone that has been processed accordingly before the surgical procedure, e.g., by inserting holes for inserting eccentrics.

Furthermore, the osteosynthesis assembly kit comprises one or preferably several eccentric connectors. Eccentric connectors are known from the field of furniture construction. They typically comprise an eccentric and a bolt, especially a screw bolt. Since the principle of the eccentric connector is also applicable to other mechanical connecting means having a head in which the eccentric can engage, in the context of the present invention, an eccentric connector is also to be understood as a combination of a screw with a screw head and an eccentric.

It is also clear from the field of furniture construction that an eccentric connector can always be constructed in at least two parts. Furthermore, a cover cap can also be advantageously provided, as it is also used analogously in furniture construction.

Due to the rotation of the eccentric during assembly under engagement with the screw or bolt head, an additional tightening of the eccentric connector is achieved, thus creating a tension screw principle. This has particular advantages in the medical field, as it brings the bone implant closer to the bone or another bone implant and additionally reduces the gap or fracture site between the two elements. This results in a firm and secure bond for osseointegration and thus promotes healing.

In addition, the assembly principle is already familiar to the surgeon due to the widespread use of the eccentric connector in other areas of application, so that the steps in the operation are virtually self-explanatory.

It is advantageous if the eccentric connector is at least in two parts, comprising a bolt or screw for insertion into a bone, having a bolt head or screw head, and an eccentric for insertion into the bone implant, wherein the eccentric has an internal helix for engagement with the bolt head or screw head.

The bone implant can be an artificial patient-specific bone implant, preferably made of a plastic or a ceramic material.

The bone implant is preferably designed as a bone plate.

The bolt can preferably be designed as a screw bolt with a tool reception.

The bolt or screw and/or the eccentric must be made of a resorbable material or of steel or titanium.

It is advantageous if the bolt or screw, the eccentric, a cover cap to cover the tool reception and/or a sleeve along the bolt has a release or active ingredient system to promote healing and/or disinfection. The cover cap can counteract unintentional loosening of the eccentric.

The bone implant has, in particular, holes to accommodate eccentrics.

The eccentrics can have one or more latching lugs in the area of the inner helix for locking the bolts during a screwing movement.

The bone implant can preferably be achieved by at least one three-point support. Therefore, the osteosynthesis assembly kit includes at least three bolts or screws and three eccentrics.

The bone implant advantageously includes one or more internal bearing surfaces for pre-centering the bone implant when it is placed on the bolts. These can be slightly oversized to match the contours of the bolts or screws or at least their screw heads or bolt heads.

The osteosynthesis assembly kit may also include a drilling template for positioning blind holes in a bone, especially in the edge of an opening in a skull cap.

Alternatively or additionally, a casting compound can be included in the osteosynthesis assembly kit for casting the eccentric with the bone implant. This can be an exposite resin, for example. Resorbable material and material with the above-mentioned release and/or active ingredient system may also be included in the casting compound.

Alternatively or additionally, it is also possible to work without casting compound, so that the eccentric connectors can be detached and the bone implant can be removed again if necessary.

The bone implant can have an advantageous multimodal structure.

The use of an eccentric connector to fix two bones, in particular at least one artificial bone implant with a natural bone, is also in accordance with the invention. Connections between a natural bone, i.e., a natural bone implant and a bone or between two bone implants are also possible.

The bone implant can therefore have a multi-modal, multi-part structure.

Individual parts or all parts of the osteosynthesis assembly kit can advantageously be designed as single-use components.

### BRIEF DESCRIPTION OF THE DRAWING FIGURES

In the following, the invention is explained in more detail by means of an exemplary embodiment with reference to the following FIGS. 1 and 2, wherein:

FIG. 1: shows an assembly system according to the invention, comprising a prefabricated skull plate and multiple eccentric connectors; and

FIG. 2: shows a known eccentric connector.

### DETAILED DESCRIPTION

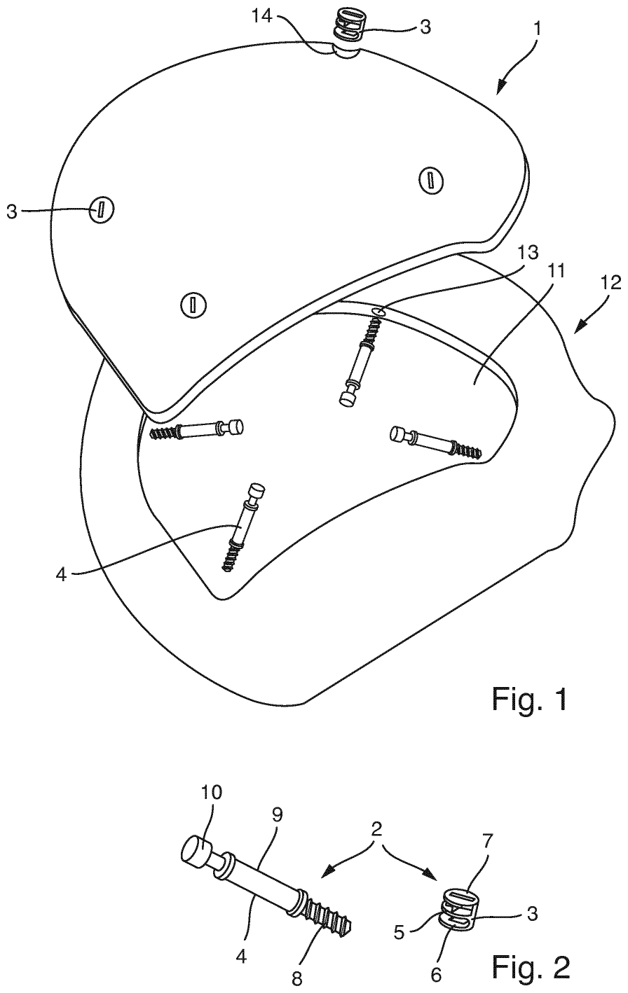

FIG. 1 shows an osteosynthesis with an artificial bone implant 1 in the form of a skull plate 1a. This can preferably be produced individually for each patient. This can preferably be done as follows, analogous to WO 2017 153 560 A1:

A) Collecting data from a patient for whom the skull plate is intended as a plate-shaped bone implant; The bone implant may consist of a single skull plate or two or more bone or bone fragments. The data may preferably be images from an imaging technique. Usually, these images are used for diagnostic imaging. In the present case, however, these images are taken independently of a diagnosis to create a model, or alternatively, they are used after a diagnosis has been made. Further details on preferred imaging techniques can be found in WO 2017 153 560 A1.

B) Creating a model based on the acquired data; The model can be created as a computer model.

C) Creating manufacturing specifications for at least one bone implant on the basis of the created model, wherein the manufacturing specifications comprise: C1) A dimensioning of the bone implant.

The manufacturing specifications are preferably created using the computer model in such a way that the part elements are dimensioned differently depending on their function. A bone plate is also somewhat thicker in one place than in another. The required material thickness and flexibility of the bone implant sections can be individually designed and manufactured by the model, which is based on patient-specific data.

D) Manufacturing of the bone implant based on the manufacturing specifications; wherein the partial elements can also be assembled with each other of the bone implant. The step of manufacturing partial elements can also include the adaptation of prefabricated elements, e.g., grinding down a preform based on a patient-specific manufacturing specification. The creation of manufacturing specifications for a bone implant is based on the created model, wherein the manufacturing specifications include the following additional information:

C2) A material selection regarding one or more materials for the bone implant,
This allows an optimal adjustment regarding a desired flexibility or rigidity of the partial elements.
C3) A selection of the number and/or position of eccentric connectors used for anchoring in the body.

FIG. 1 shows in addition to the skull plate an eccentric connector 2 comprising an eccentric 3 and a screw bolt 4. The eccentric 3 is designed cylindrically and has an internal helix 5 and a recess 6 for the insertion of a bolt head 10. Furthermore, the eccentric has a tool reception 7, e.g., a slotted reception, a cross slotted reception and/or a hexagonal reception or similar, in which a tool can engage.

By turning the eccentric 3, e.g. by 90°, 180° or more, the bolt head 10 is brought into engagement with the internal helix 5.

The internal helix 5 is designed as a double spiral in FIGS. 1 and 2, but it can also be a single spiral.

In FIG. 2, the screw bolt 4 has a screw segment 8, a bolt shaft 9, and a terminal bolt head 10.

The skull plate 1a has holes 14 to accommodate the eccentrics 3, such that the tool reception located in the eccentric 3 is accessible from the outside, i.e., from the outside of the skull cap 12.

When assembling the skull cap, blind holes 13 are first drilled into the edge of the opening 11 of the skull cap 12. The edge of opening 11 can also be pretreated to avoid undercuts, sharp corners and the like.

Then the screw bolts are inserted into the blind holes 13 in such a way that the bolt shafts 9 and the bolt heads 10 protrude radially inwards into the opening 11 of the skull cap 12.

The skull plate is then placed on the bolt shafts 9 and bolt heads 10. While in the furniture application area the furniture walls are often plugged together, in this case only the skull plate is intended to be positioned or placed, as there is no more space available. However, it is also possible to mount the skull plate on one side only, e.g., on two bolts, so that it is only placed on the other two bolts.

Furthermore, the eccentrics 3 are inserted into the holes 14 and twisted so that the internal helix 5 engages with the respective bolt head.

Finally, a cover cap (not shown) can be placed on top of the eccentric, which prevents deposits in the tool reception.

In a preferred embodiment variant, the internal helix 5 can have one or more latching lugs, as an anti-twist device for the eccentric 3.

The material of the screw bolts 4 and/or the eccentric 3 can be a metal, preferably steel, especially stainless steel, or titanium.

Alternatively, the screw bolt 4 and/or the eccentric 3 can also be made of plastic, e.g., PEEK, and in particular of a resorbable plastic, e.g., PLA. Screw bolt 4 and/or the eccentric 3 or other components such as a cover cap or a casting compound can also be made of magnesium or magnesium oxide or hydroxyapatite.

The bolt shaft 9 may have an external sleeve. This sleeve or the cover cap may be made of a resorbable plastic material, which is equipped with a release and/or active ingredient system, in particular coated or integrated. The term active ingredients or active ingredient systems refers to medicinal products. These can be e.g., antibiotics, immunosuppressants, growth hormones, or cytostatics. Release systems include all substances that are typically added to the drug, for example to control the release of the drug in the body with regard to the time and place of delivery or to preserve the drug under different conditions. Typical substances are e.g., tocopherol, which is often added as an antioxidant, or polysaccharides, e.g., chitin, for the microencapsulation of a corresponding drug. The choice of the release system also depends, among other things, on the choice of the material, i.e., the base material, from which the sleeve or cap is to be made. The eccentric 3 and/or the screw bolt 4 can also have such a drug and/or release system.

The blind holes 13 can preferably be drilled at a slight angle to allow better access to the bolts.

FIG. 2 again shows the eccentric connectors 2 in detail.

For a better fixing of the screws a hose, e.g., a steel hose with a bit, can be attached to a tool.

In the context of the present invention, screw bolts do not necessarily have to be used for the eccentric connector. Socket pins or a screw are also conceivable, for example.

On the opposite side of the bone plate, i.e., on the inside, a prefabricated bearing surface can be arranged in the contour of the screw bolt, which allows pre-centering of the bone plate when it is placed on the screw bolts.

For the positioning of the blind holes 13, a drilling aid, such as a drilling template, can also be provided in an assembly kit in addition to the skull plate and the several two-part eccentric connectors.

A tool for inserting the bolts 4 and/or a drill for the blind holes 13 can also be supplied in the assembly kit.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### REFERENCE NUMERALS

1 Bone implant
2 Eccentric connector
3 Eccentric
4 Screw bolts
5 Internal helix
6 Recess
7 Tool reception
8 Screw segment
9 Bolt shaft
10 Bolt head
11 Opening
12 Skull cap
13 Blind hole
14 Holes

1-15. (canceled)

16. An osteosynthesis assembly kit, comprising:
a bone implant; and
at least one at least two-part eccentric connector.

17. The osteosynthesis assembly kit of claim 16, wherein the at least one at least two-part eccentric connector comprises a bolt or screw for insertion into a bone, having a bolt or screw head, and an eccentric for insertion into the bone implant, wherein the eccentric has an internal helix for engagement with the bolt or screw head.

18. The osteosynthesis assembly kit of claim 16, wherein the bone implant is an artificial bone implant manufactured individually for each patient and made of a plastic, metal or ceramic.

19. The osteosynthesis assembly kit of claim 16, wherein the bone implant is a bone plate.

20. The osteosynthesis assembly kit of claim 16, wherein the bolt or screw is a screw bolt with a tool reception.

21. The osteosynthesis assembly kit of claim 16, wherein the bolt or screw or the eccentric comprises a resorbable material or of steel or titanium.

22. The osteosynthesis assembly kit of claim 16, wherein the bolt or screw, the eccentric, a cover cap for covering the tool reception, or a sleeve along the bolt has a release or active ingredient system for promoting healing or disinfection.

23. The osteosynthesis assembly kit of claim 16, wherein the bone implant has holes for accommodating eccentrics of the at least one at least two-part eccentric connector.

24. The osteosynthesis assembly kit of claim 27, wherein, in a region of the internal helix, the eccentric has one or more latching lugs configured to latch the bolt during a screwing movement.

25. The osteosynthesis assembly kit of claim 16, wherein the osteosynthesis assembly kit comprises at least three bolts or screws and three eccentrics.

26. The osteosynthesis assembly kit of claim 16, wherein the bone implant has internal bearing surfaces for pre-centering the bone implant when the bone implant is placed on the bolts or screws.

27. The osteosynthesis assembly kit of claim 16, further comprising:
a drilling template configured for positioning blind holes in an edge of an opening in a skull cap;
a tool for inserting the bolts or screws; or
a drill.

28. The osteosynthesis assembly kit of claim 17, wherein the eccentric is detachable.

29. The osteosynthesis assembly kit of claim 16, further comprising:
a casting compound with which the at least one at least two-part eccentric connector can be cast after assembly.

30. A method of using an osteosynthesis assembly kit, the method comprising:
inserting an eccentric into a bone implant;
inserting a bolt or screw into a skull cap;
coupling the bone implant to the skull cap by inserting the bolt or screw into the eccentric and rotating the eccentric to lock the bolt or screw in the eccentric.
